(19) United States
(12) Patent Application Publication
(10) Pub. No.: US 2012/0126024 A1
(43) Pub. Date: May 24, 2012

(54) METHODS AND SYSTEMS TO DELIVER VOLATILE COMPOUNDS

(76) Inventors: Maurice M. BOYD, Oakland, FL (US); Ronald D. NEUMAN, Auburn, AL (US); Andrew B. SHELTON, Auburn, AL (US); Wiley M. CAUTHEN, Winter Garden, FL (US); Ralph H. ZEE, Auburn, AL (US)

(21) Appl. No.: 13/300,067

(22) Filed: Nov. 18, 2011

Related U.S. Application Data
(60) Provisional application No. 61/415,035, filed on Nov. 18, 2010.

(51) Int. Cl. A61L 9/04

(52) U.S. Cl. 239/6; 239/59

(57) ABSTRACT

Methods and systems for biological control of pests and pest-borne diseases, such as in agriculture, include vapor delivery systems with (a) a reservoir for holding the volatile compound(s), typically in liquid form, to be released into the atmosphere, (b) a means of supplying the reservoir with volatile compound(s), (c) one or more passive flow control nozzles attached to the reservoir, wherein each nozzle is fitted with a permeable polymeric membrane through which the volatile compound permeates and is emitted from the outer membrane surface into the atmosphere, and wherein the individual nozzles are adapted to deliver a specific predetermined release rate for each intended application, and (d) strategic deployment of nozzles (i.e., number, location, spacing, height, etc.) to achieve performance requirements for intended applications ranging from a single plant to large fields for specific ambient conditions.

### CROSS-REFERENCE TO RELATED APPLICATION

This application claims the benefit of U.S. Provisional Patent Application Ser. No. 61/415,035, filed Nov. 18, 2010, the entirety of which is hereby incorporated herein by reference for all purposes.

### TECHNICAL FIELD

The present invention relates generally to methods and systems for the delivery of volatile compounds into the atmosphere, and more particularly to the controlled delivery of volatile compounds at continuous, predetermined, substantially constant and sustainable release rates to yield a product dispersion of ultra-low effective concentrations in open environments over very long time periods, such as up to and exceeding one year.

### BACKGROUND

It is known that plants, in response to insect attacks or leaf damage, can release volatile natural compounds that activate various plant defensive mechanisms. For example, herbivore-induced plant volatiles (HIPV) are believed to provide induced resistance (IR) or systemic acquired resistance (SAR) that help plants to both resist and recover from diseases (Agrawal et al., 1999). One of the emitted HIPVs, methyl salicylate, may be involved in plant-to-plant communication whereby a neighboring plant builds up its immune response if a stressed plant emits the volatile chemical (Karl et al., 2008). Additionally, it is known that methyl salicylate can function as an attractant for beneficial insects to keep pest populations under control (James, 2003).

As early as 1989 (Auger et al.), it was known that some plant species produce volatile sulfur compounds such as dimethyl disulfide (DMDS). In subsequent studies (Auger et. al, 1994), it was proposed that DMDS offers great potential in plant protection and in seed storage systems as a replacement fumigant for methyl bromide because of its known lethality for many insect species, the mechanism of which has recently been clarified (Dugravot et al., 2003). Interestingly, the active components in garlic are also sulfur compounds and have been used as a natural insect repellent and insecticide (Huang et al., 2000). More recently, it was shown that DMDS is formed in crushed guava leaves, but not in intact leaves, and thus it appeared to be produced in response to wounding or mechanical injury of the plant leaves (Rouseff et al., 2008). Furthermore, the volatiles released by guava leaves have been of intense interest since it was reported that guava grown in proximity to citrus trees in Vietnam had a plant protective or repellent effect against the Asian citrus psyllid (Beattie et al., 2006).

The art of emitting or releasing volatile compounds and substances has a long history. There are a variety of devices or systems described in the patent literature to evaporate or dispense volatile compounds or compositions into the atmosphere (Emmrich et al., U.S. Pat. No. 6,582,714; Kvietok, et al., U.S. Pat. No. 7,481,380). Materials commonly delivered include fragrances, deodorizers, disinfectants, insecticides, air fresheners, etc. Delivery dispensers typically can be categorized as “active dispensers” or “passive dispensers”. For example, a common active dispensing device is the aerosol spray dispenser that propels minute droplets of a volatile composition into the air. Active dispensers include various types of sprayers that operate by pressure, air displacement, or pump action. There are other dispensers that require an energy source. For example, devices or articles that dispense insecticide vapors often utilize the heating or burning of a liquid or solid substance to evaporate the active ingredients. Other dispensing methods include substrates such as paperboard or fabrics impregnated with volatile active ingredients, gelatinous materials that as they dry and shrink release a volatile compound into the air, and micro-encapsulated substances that achieve a slow release of volatile active ingredients. Evaporative surface (non-aerosol) devices typically utilize a wick or porous surface that provides a large surface area from which volatile liquid material can more quickly evaporate passively into the air. Attempts at improvements on the shortcomings of dispensing devices have included combining elements of both active and passive dispensers into a combined device.

Some vapor (or gas) dispensing devices have employed permeation membranes, but their intended usage generally has been focused on more specialized applications. For example, permeable membranes have been used in the production of calibration samples for gas or liquid analyzers, such as in tube devices (O'Keeffe, U.S. Pat. No. 3,412,935) and in devices having improved membrane permeability characteristics (Chand, U.S. Pat. No. 3,856,204). An apparatus used for the treatment of honeybee colonies for different honeybee diseases employed microporous membranes (Orth, U.S. Pat. No. 6,820,773). Vapor-permeable membranes also have found use in a fragrance product (Obermayer and Nichols, U.S. Pat. No. 4,356,969) and in a time-temperature indicator for monitoring the shelf lives of perishable articles (Patel, U.S. Pat. No. 4,195,058).

In an application for pest control, a sex pheromone has been dispensed by using a plastic bag through which the pheromone compound permeated the bag walls and was released as a vapor to the atmosphere (Kauth and Darskus, German Pat. No. 28 32 248; Kauth et al., German Pat. No. 29 45 655). Another approach to the control of a pest employed a capillary tubing of a polymeric material filled with a vaporizable substance, such as a pesticide, fungicide or sex pheromone, which permeated the tube walls and was released to the atmosphere; this dispensing body had good shape-retainability by integrating side-by-side a metal wire with the capillary tubing (Ohno, U.S. Pat. No. 4,600,146). A method for simultaneously controlling the rates of concurrent vapor release of two specific classes of sex pheromone compounds involved mixing in a unique proportion to achieve an overall solubility parameter and enclosing the liquid mixture in a permeable container such that two pheromone compounds permeated the wall and were dispensed into the atmosphere as a vapor (Yamamoto et al., U.S. Pat. No. 4,734,281).

The previously available methods, devices and systems were usually limited to dispensing vapors within defined spaces, for example, a room or the area in the immediate vicinity of a device. These vapor dispensers are generally known in the art to provide inadequate effectiveness in larger, more open spaces, especially in large volumes of moving air. Another difficulty is that they are not able to effectively dispense volatile compounds at a sustainable rate over long periods of time. Furthermore, another undesirable characteristic is that there is often an initial burst of vapor followed by a continuous intensity decline, rather than a delivery of vapor at a rate essentially constant with time.

Thus, there is a need for effective methods and systems for the controlled delivery of volatile compounds in large open areas such as agricultural fields, citrus groves and orchards. However, there are serious deficiencies in the practical, effective deployment of volatile compounds in open field environments. The design goals include: (1) substantially constant and consistent, predetermined rate of delivery, (2) sustained rate of delivery over extended time periods of growing seasons up to and in some instances exceeding one year, and (3) controlled rate of delivery that provides ultra-low effective concentrations under open field conditions.

The present invention generally meets these goals for the delivery of volatile compounds in a wide variety of practical applications. As one example, the present invention holds great promise to make effective biological strategies available in the fight to control the spreading of serious invasive plant diseases, such as the citrus greening disease (Zaka et al., 2010) and the potato zebra chip disease (Miles, 2010).

It is to the provision of devices and methods for delivering volatile compounds meeting these and other needs that the present invention is primarily directed.

### SUMMARY

In example embodiments, the present invention comprises devices and methods that provide controlled delivery of one or more volatile compounds at substantially continuous, predetermined, substantially constant and sustainable release rates to yield a product dispersion of ultra-low effective concentrations in the ambient atmosphere over long time periods.

In one aspect, the present invention relates to a vapor delivery system that provides unique advantages in an open atmosphere having large volumes of moving air, wherein the system can provide controlled delivery or release rates at strategic locations which, in turn, yield effective concentrations of volatile compound(s) in open field environments.

In another aspect, the invention relates to methods of preparing and using vapor delivery systems for biological control of pests and pest-borne diseases in a wide variety of practical applications ranging from the protection of agricultural products such as fruits, vegetables, trees and flowers to the protection of people, pets, livestock, stored grains and foodstocks.

Briefly described in general terms, in a first preferred form the invention comprises a vapor delivery system including (a) a reservoir for holding the volatile compound(s), typically in liquid form, to be released into the atmosphere, (b) a means of supplying the reservoir with volatile compound(s), (c) one or more passive flow control nozzles attached to the reservoir, wherein each nozzle is fitted with a permeable polymeric membrane through which the volatile compound permeates and is emitted from the outer membrane surface into the atmosphere, and wherein the individual nozzles are adapted to deliver a specific predetermined release rate for each intended application, and (d) strategic deployment of nozzles (i.e., number, location, spacing, height, etc.) to achieve performance requirements for intended applications ranging from a single plant to large fields for specific ambient conditions.

In one preferred form the invention comprises an apparatus for applying a controlled amount of a volatile compound to an open outdoor area or to a similarly open indoor area over a long period of time. The apparatus includes a compound-dispensing module comprising a container for containing a volume of the volatile compound. A permeable membrane is provided at least partially capping the volume containing the volatile compound and permitting the volatile compound to pass therethrough by permeation at a substantially constant rate. A partially open cover is provided having openings formed therein and being positioned over the permeable membrane. Preferably, the membrane is a polymeric membrane. Optionally, the apparatus can be configured as a stand-alone container. Also optionally, the apparatus can be configured as a distributed network of modules, with the modules being linked by supply pipes to deliver the volatile compound to the modules. The volatile compound can be moved through the pipes by pumps, by gravity, or by other means.

The present invention also can take the form of a system for applying a controlled amount of a volatile compound to an open outdoor area or to a similarly open indoor area over a long period of time. The system includes a plurality of spaced-apart compound-dispensing nodes, each compound-dispensing node having a permeable membrane permitting the volatile compound to pass therethrough by permeation at a substantially constant rate. A main supply reservoir is provided for containing a supply of the volatile compound and a plurality of conduits extend to the membranes for delivering the volatile compound from the main supply to the membranes.

In another form, the present invention comprises a method for effecting a controlled release of a volatile compound over time at or adjacent a desired location. The method includes the steps of providing a reservoir for containment of a volatile compound to be released, the reservoir including a permeation membrane, and containing the volatile compound within the reservoir. The method also includes the step of positioning the reservoir at or adjacent the desired location.

One ready application (among many) for the invention is to treat citrus trees or citrus groves. To that end, the invention also includes the method of treating a citrus tree at risk of disease, infection, or infestation, etc. The method includes the step of placing a quantity of a selected volatile compound in a container, the container having a permeable membrane adapted and configured to allow the volatile compound to permeate therethrough at a low, substantially constant rate over a long period of time. The method also includes the step of attaching the container to the citrus tree to apply the volatile compound to the citrus tree and to its immediate surroundings and leaving the container on the citrus tree for two months or more to deliver the volatile compound to the citrus tree for two months or longer.

The present invention is based on the concept of a practical means to mimic the release of volatile compounds from the surface of plant leaves, and then to employ those volatile compounds having known benefits in practical applications, such as biological pest control and biological crop disease management in agriculture, forestry, horticulture and floriculture as well as landscape and ornamental plants. Among the benefits of biological control, especially when large-scale application systems, such as those of the present invention are implemented, will be reduced usage of various pesticides with significant reduction in pesticide residues in crop products, thereby also yielding overall environmental and food product safety benefits.

Preferably, the systems and methods of the present invention provide a substantially continuous, consistent and sustainable rate of delivery over extended periods of time, and achieve an ultra-low effective concentrations in the ambient atmosphere of open environments subject to moving air currents. The approach employed in the present invention employs passive flow control nozzles with permeable membranes. The science underlying membrane technology is well known to those versed in the art.

The present invention represents a break-through in the art of controlled delivery of volatile compounds in several aspects. Indeed, the methods and vapor delivery systems of the present invention described herein can: provide substantially continuous, constant and sustainable rates of delivery over entire growing seasons that may last many months; provide controlled rates of delivery at strategic field locations which yield effective concentrations in open environments on the order of parts per billion, or even lower, per application requirements; permit fine-tuning or adjustment of the rate of delivery to a predetermined rate for specific applications; provide flexibility to utilize diverse groups of volatile compounds having vapor pressures that range over several orders of magnitude; and permit deployment of advanced bio-control configurations, such as combinations of repellents, attractants, interference agents, and immune enhancement promoters in agricultural fields, citrus groves, and other plantings.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1A is an exploded cross-sectional view of an example form of a vapor delivery system according to a first preferred form of the present invention.

FIG. 1B is an exploded cross-sectional view of an example form of a vapor delivery system according to a second preferred form of the present invention.

FIG. 1C is an exploded cross-sectional view of an example form of a vapor delivery system according to a third preferred form of the present invention.

FIG. 2 is a schematic view of another example vapor delivery system according to a fourth preferred form of the present invention, deployed in a notional (agricultural) grove plot plan.

FIG. 3A is a schematic illustration of another example vapor delivery system according to a fifth preferred form of the present invention.

FIG. 3B is a schematic illustration of another example vapor delivery system according to a sixth preferred form of the present invention.

FIG. 3C is a schematic illustration of another example vapor delivery system according to a seventh preferred form of the present invention.

FIG. 3D is a schematic illustration of another example vapor delivery system according to a eighth preferred form of the present invention.

FIG. 4 is a graph schematically depicting a comparison of the delivery rate of a volatile natural compound versus time for one tested embodiment of the present invention in comparison to the performance of a prior art technology.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The term “open environments” as used herein, refers to environments subject to large-scale moving air currents as opposed to restricted spaces such as a room, closet, etc. Distinction, however, must be made between open local environments and open field environments. The former refers to relatively small air volumes including but not limited to single trees, individual landscape or garden plantings, and near outside door or entryways. Enclosures for storage of foodstuff or feeds within silos can also be considered as open local environments because they are not perfectly sealed, but leak air. These situations are in marked contrast to open field environments that include large agricultural and floricultural fields, citrus groves, orchards, vineyards, etc. Examples of intermediate-scale environments include home gardens, nurseries, and greenhouses.

The term “volatile compounds” as used herein, refers to organic compounds or materials that are vaporizable at ambient temperature and atmospheric pressure without the addition of energy by some external source. Any suitable volatile compound in any form may be used. Volatile liquids composed of a single volatile compound are preferred for large-scale application, but volatile solids can also be used for some specialized applications. Liquids and solids suitable for use may have more than one volatile component, and may contain non-volatile components. The volatile compounds may be commercially pure or blended and, furthermore, may be obtained from natural or synthetic sources.

In the context of the present invention, the term “vapor delivery systems” as used herein, refers to those vapor delivery systems that are based on passive flow control nozzles that utilize permeable polymeric membranes. There are two primary preferred systems or approaches in the present invention—fixed supply, stand-alone units and replenished, distributed systems (such as replenished by gravity or by pumps). The vapor delivery system with a fixed supply is used to deliver volatile compounds in either open local environments or open field environments. On the other hand, the pumped delivery system with a piped supply distribution header is uniquely suited for applications in open field environments. These systems are classified as passive systems since the vapor that results from volatilization at the membrane surface is dispensed by stagnant diffusion and/or random air circulation over the flow control nozzles. The pump is used to move the volatile compound from a storage reservoir to the passive flow control nozzles.

The term “passive flow control nozzles” as used herein, refers to a permeable membrane and a retaining structure that together constitute the nozzle that controls the rate of delivery of volatile compounds. The flow control nozzle can take various geometrical forms (flat or cylindrical) depending on the configuration of the membrane. The flat membrane is used with a retaining band or fitting that allows control of the exposed membrane area. The pumped vapor delivery system can be designed such that the piping itself or sections of the piping, if constructed of select polymeric material, are able to function as passive flow control nozzles.

For a more complete understanding of the present invention and its potentialities, it is useful to briefly examine membrane permeation processes in the context of the fixed-supply vapor delivery system of the present invention. In vapor permeation, the vapor of the volatile compound is in contact with a permeable solid membrane. The molecules in the vapor phase first dissolve in the membrane, then diffuse through the membrane, and finally diffuse and mix in the open environment. In the case of a vapor in equilibrium with its liquid phase, the vapor pressure of the volatile compound—and thus the release rate—will be constant for a given temperature. In liquid permeation, the liquid phase instead of the vapor is in contact with the permeable membrane. The molecules of the liquid phase dissolve in the membrane at higher concentration before diffusing through the solid to the open environment.

Prior to a detailed description of the invention, it is beneficial to examine the design considerations underlying the development of a vapor delivery system to achieve the requirements set forth earlier in the ‘Summary of the Invention’. Factors that must be considered to design a vapor delivery system for a given volatile compound include the membrane material and type, membrane surface area, membrane thickness, contacting phase, temperature, pressure and membrane stability. Knowledge of the prevailing airflow conditions at the site of deployment of a vapor delivery system is also desirable for optimal system design.

To validate the performance of a polymeric permeation-based vapor delivery system using a selected volatile compound, laboratory tests under controlled conditions were performed to evaluate whether the concept is both applicable to the needs and that a sustained, substantially constant release rate can be maintained for long periods of time. Permeation membranes made from off-the-shelf materials were tested to evaluate the release rates for a range of membrane surface areas and thicknesses. Evaluations using both liquid and vapor phases in contact with the membranes were performed.

The performance of a membrane is strongly dependent on its chemical and physical properties. The chemical interactions between membrane material and volatile compound determine the solubility and permeability of the molecules diffusing in the membrane. In the case of polymeric membranes, the molecular weight, crystallinity, density and dimensional stability are also important considerations in membrane selection. Furthermore, the phase of the volatile compound in contact with the membrane, either vapor or liquid, is an important design parameter because the concentration adjacent to the membrane surface establishes the driving force for diffusion through the membrane. The membrane thickness is another important design parameter since the release rate is also determined by the resistance to diffusion in the membrane, and the resistance can be controlled by selection of material, selection of thickness and/or design of composite membranes.

The membranes employed in the present invention are preferably nonporous, homogeneous solids and are selected to yield the desired release rate for the specific volatile compounds. The membrane composition is typically that of a solid polymeric material, either natural or synthetic. Some selected examples of polymeric membranes include but are not limited to polypropylene, polyethylene, copolymers of ethylene or propylene, cellulose acetate, polyacrylonitrile and copolymers of acrylonitrile, ABS, polyesters including polyethylene terephthalate, acetal copolymers, polycarbonate, poly(4-methylpentene-1) and various fluorocarbons. For some applications, the use of a porous membrane may be advantageous for fine-tuning a release rate. Such a membrane is designed to be vapor permeable and liquid impermeable. As an example, a microporous membrane with select hydrophobic and oleophobic properties may be substituted for a nonporous membrane to provide an increase in release rate or to modify a release rate for a given volatile compound to achieve a designed release rate.

It is well known that temperature affects the release rate of volatile compounds, and the present invention exploits this effect in a novel manner in some applications. The effect of temperature on the release rate can arise from two sources, one is related to the temperature dependence of the intrinsic permeability of membranes, and the other is the effect of temperature on vapor pressure. In one specific application, the release rate is designed for an average rate over a growing season or part of a growing season when pests or insects are most active. In this situation, the passive flow control nozzles are “self regulating” in that the release rate automatically increases with higher temperatures coinciding with increased pest activity; therefore more volatile compound is made available for control. On the other hand, less volatile compound is naturally released during lower temperatures when pests are less active. In applications where temperature variation has an undesirable impact on the release rate, other parameters can be adjusted to compensate. Pressure is one such parameter that can be monitored and adjusted in real time to fine control the release rate for many applications.

The particular details of the open environment surrounding the vapor delivery system play an important role in the overall performance and cost-effectiveness of the system and therefore influence its physical configuration. The scale of the system is defined by its parameters such as spatial extent, total release rate, and nozzle density, and correlates with the scale of the environment over a range from local to field. Permanency and maintainability of the installed vapor delivery system is also a consideration, and depend on the temporal scale of the intended application. Many volatile compounds are highly diffusive in air, and the motion of the air is driven by forced and natural convection processes and influenced by boundaries and structures. For a specific release rate and point of release, the combination of convective properties of the flow and diffusive properties of the vapor is the determining factor in the concentration of the volatile compound within the open environment. Therefore, the system should allow for release point(s) that can be (re) configured depending on airflow conditions or optimized over a range of airflow conditions. The system should also allow for the release of different volatile compounds in different regions of the open environment when a specific application warrants such a complementary combination.

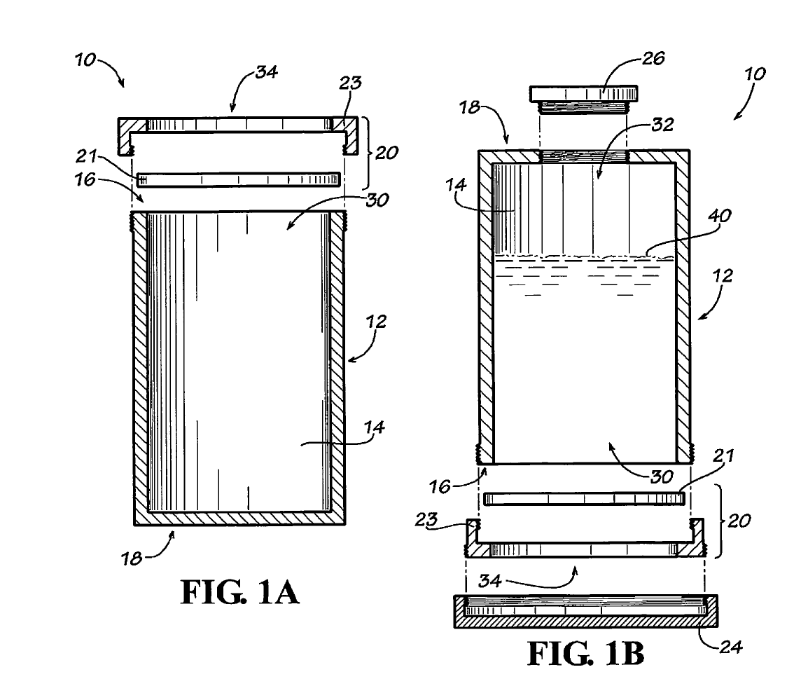

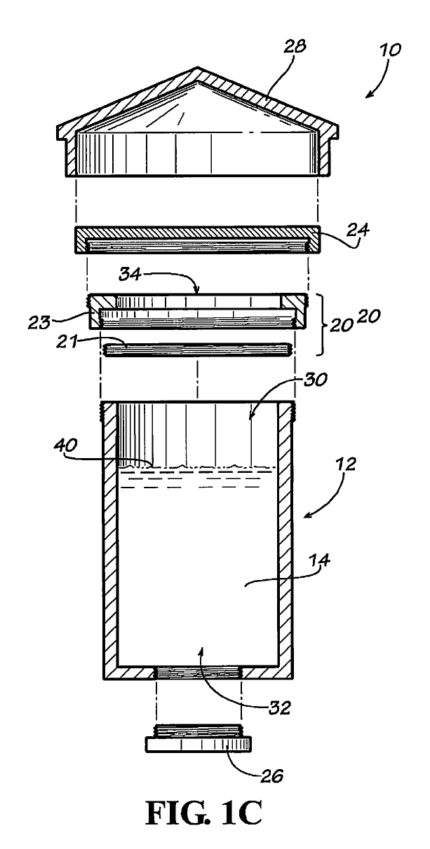

FIG. 1A depicts a fixed-supply vapor delivery system (fixed system) 10 according to an example form of the invention. In general, the fixed-supply vapor delivery system 10 comprises a reservoir or container 12 and passive flow control nozzle 20. In example embodiments, the container 12 defines an internal elongate cavity 14 that stretches from a first end 16 comprising an opening 30 to a second end 18 (see FIG. 1A). The passive flow control nozzle 20, in its simplest form, comprises a flat permeable membrane 21 and a retaining band fastener 23. The retaining band fastener 23 has an open area or aperture 34 of predetermined dimensions through which the vapor emitted from the membrane passes prior to mixing with the ambient atmosphere. The permeable membrane 21 is positioned between the reservoir 12 and the retaining band 23 to attach the membrane to the reservoir when the band is removably coupled to the first end 16 of the reservoir. In alternate embodiments, the second end 18 of the container 12 can comprise a supply port 32 used for filling (and refilling) the reservoir with the volatile liquid upon the removal of a filler plug 26 (see FIGS. 1B-C). For example, the reservoir depicted in FIGS. 1B-C contains a vaporizable liquid in equilibrium with its vapor as shown schematically by the existence of a vapor-liquid interface 40. Additionally, a sealing cap 24 can be removably coupled to the retaining band 23 when shipping the fixed system with volatile compound or when not in active use. When the fixed-supply vapor delivery system 10 is in active use, a debris cover 28 may be mounted to the retaining band 23 if site conditions warrant. For any particular volatile compound, the rate of release and the resulting local vapor concentration depend on the choice of the permeable membrane used, its thickness, and area of exposed surface. The lifetime of a specific vapor emission is determined by the quantity of volatile liquid in the reservoir, and thus a sustained rate of release for an extended period of time can be achieved by increasing the capacity of the reservoir. The reservoir is made of a material impervious and typically non-reactive to the contents of the reservoir. In some applications it may be desirable to employ materials that are biodegradable.

FIG. 1B illustrates the form of the fixed system with liquid in contact with the permeable membrane. FIG. 1C illustrates the form of the fixed system with vapor in contact with the permeable membrane. The resulting release rate is greater than that when the vapor phase is in contact with the membrane. Furthermore, it may not be necessary to use a debris cover to protect the membrane from harmful contamination. It may be advantageous to maximize permeation area by replacing a portion or all of the reservoir with a cylindrical permeable membrane (not shown).

The uniqueness of the fixed system is its flexibility, portability, and versatility. The fixed system is a self-contained package that is easily installed and requires no power source to function effectively. It is capable of serving multiple applications such as single plants, forests, landscaping, gardens, nurseries, open fields, grain and foodstuff storage, human and animal pest control, and fragrance enhancement. Its performance in dispersing volatile compounds makes advanced biological control available to a wide range of type and size of applications.

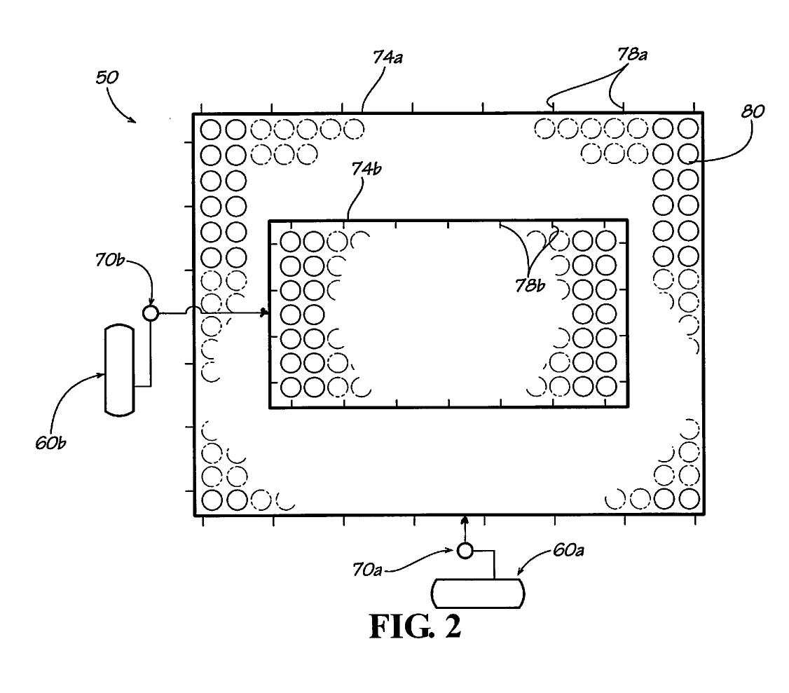

In another preferred embodiment of the present invention, FIG. 2 illustrates a pumped-supply vapor delivery system (pumped system) 50. The pumped system 50 comprises a supply storage reservoir 60a and a supply pump 70a. The supply storage reservoir 60a can contain and supply the volatile compound that is to be released from the passive flow control nozzles into the atmosphere. The supply pump 70a, in fluid communication with the reservoir 60a, is further engaged to one or more supply distribution headers 74a comprising a plurality of nozzles 78a. The pumped system 50 is uniquely suited for applications in open field environments, especially large acreages of plants or crops that require the controlled release of a volatile compound from multiple sources strategically placed in order to provide a predetermined effective concentration in the ambient atmosphere. This approach to the delivery of volatile compounds makes possible the practical application of advanced biological control strategies in agricultural fields, groves or orchards.

In yet another preferred embodiment of the present invention, multiple pumped systems are employed in an open field environment. FIG. 2 schematically illustrates the deployment of two pumped systems for the implementation of a specific biological control strategy. The multi-system configuration additionally shown in FIG. 2 permits the release of volatile compounds for a bio-control strategy hereafter called a “repellent-attractant strategy” or a “push-pull strategy”. In the model grove plan shown in FIG. 2, the piping of the first pumped system for the supply distribution header 74a is situated at the perimeter or border to release a repellent compound for the purpose of repelling or keeping pests from entering the grove and, hence, protecting the grove plantings 80 from attack by specific pests or pests in general. The second pumped system comprising a supply storage reservoir 60b, a supply distribution header 74b, and multiple nozzles 78b is located within the interior of the grove. The second pumped system can dispense an attractant compound which attracts beneficial insects or organisms and/or assists in the promotion of inducing resistance that helps plants resist and recover from diseases.

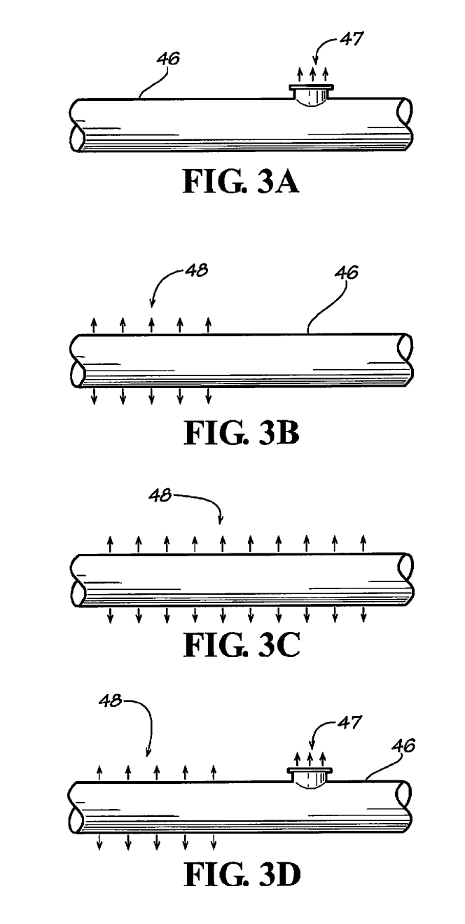

There are at least four configurations of passive flow control nozzles that can be utilized to release volatile compounds in pumped systems. FIG. 3A illustrates a section of the pumped system consisting of impermeable piping 46 and flat membrane nozzles 47. FIG. 3B illustrates a section of the pumped system consisting of alternating lengths of impermeable piping 46 and cylindrical membrane nozzles 48. FIG. 3C illustrates a pumped system wherein the entire piping of the supply header consists of a cylindrical membrane nozzle 48. FIG. 3D illustrates a combination of the above configurations.

For a specific volatile compound, the flexibility associated with the design of a vapor delivery system for a particular application permits virtually any desired system release rate to be achieved in practice. This is accomplished by diligent analysis of the specific application requirements and then proper selection of the membrane material(s), type and thickness and the membrane surface area via a combination of flat and cylindrical flow control nozzles that are strategically located within an open field environment, considering the local ambient conditions, to yield an effective concentration of the volatile compound in the ambient atmosphere. The design of a vapor delivery system is not limited to only one membrane material. Fine tuning of the system release rate can also be achieved by the use of more than one membrane material and/or membrane type.

Another preferred embodiment of the present invention includes the use of any number of pumped systems to release any number of volatile compounds for any combination of repellents, attractants, interference agents, immune enhancement promoters, or other volatile chemicals for any purpose that may or may not be related to the implementation of advanced biological control strategies.

In still another preferred embodiment, fixed systems can also be employed in open field environments either alone or in combination with pumped systems. The piping of the supply distribution header in pumped systems can also function as a passive flow control nozzle, if its chemical and physical properties are properly considered for the volatile compound used. The passive flow control nozzle, as defined earlier, consists of a flat permeable membrane connected to a retaining structure, such as a retaining band or a fitting. In the case of a permeable pipe, structure is built into the cylindrical pipe wall (or membrane), and as such, the piping itself can be considered to be a passive flow control nozzle. The relevant surface area to be used in calculations of the actual release rate of the volatile compound is the surface area of the length of piping employed as the passive flow control nozzle.

The present invention has many advantages, one of which is the flexibility it offers in the design and engineering of vapor delivery systems that release and control volatile compounds in open environments. This flexibility originates in the passive flow control nozzles employed by the present invention. The passive flow control nozzle approach permits design of a vapor delivery system that emits a predetermined release rate for a specific application of a volatile compound. It should be emphasized that the release rate from a nozzle and a system may be different. The total release rate of a vapor delivery system is the sum of the individual release rates from the nozzles making up the system as a whole. However, a system may use a single nozzle.

### EXAMPLE APPLICATIONS

One ready application of the invention is to repel Asian citrus psyllids and provide annual protection for citrus “resets”, new plantings, young trees (4-7 years) and citrus groves against Huanglongbing (HLB) disease (and other citrus diseases as appropriate). A citrus reset is a replacement for a single existing tree. Typically, it is about 18″ high. As presently considered, one would affix one fixed system per reset during the first year after planting. The citrus resets do not produce fruit in the first three years. However, in years 2 and year 3, one may be inclined to affix more than one fixed system to the growing reset. Effective control of Asian citrus psyllids may require a multiple or plurality of fixed systems according to the present invention, per individual reset.

In the context of citrus, a “new planting” is when one plants an entire new grove (or large area), typically with the above 18″ high new plantings. Once again, the plantings are not productive for the first three years of growth. A “young tree” or young trees are productive trees of 4-7 years of age. They are fruit bearing and continue to grow in height and width. Thus, a plurality of fixed passive control systems or a pumped system will often be required. The number and strategic location of passive flow control nozzles required will be based on an engineering design for a particular site and grove. A “grove” typically consists of a large array of productive mature trees whose age is eight or more years. The present invention can provide perimeter control, grove control or combination control of repellents, attractants and various promoters or interference agents (e.g., mating and reproduction interference, plant immune enhancements, etc.).

HLB or citrus greening disease is a serious threat to cultivation of citrus crops. The Asian citrus psyllid, Diaphorina citri Kuwayama (D. citri), is the primary vector in citrus of the bacteria Candidatus liberibacter asiaticus and Candidatus liberibacter americanus. These bacteria are presumed to be responsible for HLB disease. The range of D. citri has expanded into citrus production areas throughout the world, threatening entire citrus groves on a regional scale, thereby making HLB one of the most serious threats to cultivation of citrus worldwide (Halbert and Manjunath, 2004).

Control of the Asian citrus psyllid is critical to the citrus industry. Current efforts to control D. citri populations primarily rely on application of broad-spectrum insecticides by area-wide aerial spraying programs. However, numerous applications of pesticides, often eight to ten times annually in Florida, cannot be indefinitely sustained. There are serious issues with D. citri developing pesticide resistance, the build-up of environment contaminants, the unintended elimination of natural biological control agents, and pesticide residues in citrus products.

The HLB situation is virtually a crisis and cries out for new tools to control of D. citri. Fortunately, there are potential attractive alternatives such as pest repellents based on plant volatiles or plant-derived essential oils. Although there has been significant progress on repellent research in scientific laboratories, a practical repellent system (that includes both repellent and a delivery apparatus) has not yet been developed. One key bottleneck in moving this research forward has been the lack of a slow-release device that maintains the volatile repellent above a behaviorally active threshold for extended periods of time as long as 150-200 days (Onagbola et al., 2011).

The present invention can deliver volatile repellent compounds in either open local (single tree) or open field (grove) environments and help address this citrus crisis. One application concentrates on protecting citrus resets that are in the life stage of a tree that is most vulnerable to disease. If the growers cannot successfully grow resets to productive maturity, the industry is headed to extinction because of the need to plant new groves and to replace trees removed from existing groves due to canker, blight, black spot, HLB and other impacts. One potential impact of the present invention is to allow citrus resets to be grown to productive maturity without frequent use of pesticides. This allows the growers to again prudently invest in resets to give new life to the industry, stabilize and then grow the production of citrus with the resulting economic benefits to the growers and to the customers of citrus products that have reduced pesticide residue.

It is expected that psyllid control can be achieved with greatly reduced use of pesticides for citrus resets (and new plantings) by employing the present vapor delivery apparatus that releases volatile repellent which is behaviorally efficacious under field conditions over entire (annual) growing seasons. It is also expected that psyllid control can be achieved with greatly reduced use of pesticides for citrus young trees by employing the present vapor delivery apparatus that releases volatile repellent which is behaviorally efficacious under field conditions over entire (annual) growing seasons. It is also expected that psyllid control can be achieved with greatly reduced use of pesticides for developing productive citrus groves with a perimeter (vapor) barrier to repel the influx of psyllids into groves. This outcome will favorably impact the citrus growers and provide them an alternative or supplement to the numerous repeated pesticide spraying now used. Finally, it is expected that psyllid control can be achieved with greatly reduced use of pesticides within the interior of mature large groves. This can be achieved by combinations of repellents, attractants, immune enhancement promoters, mating disruption (pheromones), reproduction interference, etc. Furthermore, this is accomplished by the following strategy: The first distributed control system or pumped system is situated at the perimeter or border to release a repellent compound for the purpose of repelling or keeping pests from entering the grove. The second pumped system is located within the interior of the grove and dispenses an attractant compound with attracts beneficial insects or organisms, or attracts psyllids to a trapping or killing site and/or assists in the promotion of inducing resistance that helps plants resist and recover from diseases. Thus, there can be any combination of fixed control systems and pumped systems or distributed control systems to accomplish any desired combined purposes of protection, enhancement, etc.

The present vapor delivery system/apparatus can be used for control of pests and pest-borne diseases in a wide variety of practical applications ranging from the protection of agricultural products such as fruits, vegetables, trees and flowers. Among the benefits, especially when large-scale application systems are implemented, will be reduced usage of various pesticides with significant reduction in pesticide residues in crop products, thereby also yielding overall environmental and food product safety benefits.

The present invention has the potential for use in Integrated Pest Management (IPM) systems to protect forest and urban trees from native and exotic pests. In the United States. successful forest management is not possible without trapping programs capable of detecting low-density populations of target species. The invention holds excellent promise as a replacement method for the currently used bait dispersion methods employed in insect monitor trapping and tree protection.

The southern pine beetle has caused unprecedented losses to southern pine trees in recent years. Forestry management programs use various synthetic pheromones and terpenes of species being monitored as a sampling tool. A more effective trapping method based on chemical lure dispersal is economically important for protecting both the forests and urban trees from native pests and exotic invaders. In practice, each trap is baited with a conventional lure (e.g., turpentine/ethanol, SPB-Sirex lure, frontalin lure). Typically, chemical lures are often replaced weekly to restore their effectiveness in the field. The present invention should reduce the economic cost by greatly decreasing the frequency of lure replacement as well as providing a more constant dispersal rate of application.

The present invention will undergo field trials in the southern pine beetle spring monitoring program. The chemicals that can be employed in the invention for forestry applications include lures such as frontalin, turpentine, ethanol, alpha-pinene, beta-pinene, verbonone, as well as the repellent 4-allyl anisole, but are not limited to these chemical compounds.

The potato industry has a parallel threat similar to that of the citrus industry that is spread by a similar psyllid. The impact on an Irish potato is a discoloration in streaks such that potato chips made from these potatoes appear with streaks of discoloration called “zebra chips”. The systems, both fixed and distributed systems (including pumped and gravity-fed systems), with the correct volatile compound(s) should be an excellent way of repelling these psyllids.

Another food industry that faces threats from insects and insect-vectored diseases is the avocado industry. Using the fixed and distributed systems to disperse the correct volatile compound(s) should give this industry a new tool to fight the threat cause by insects and insect vectored diseases.

Insects such as mosquitoes, fleas, flies and ticks transmit many human diseases that can be debilitating and deadly. Using the fixed and distributed passive control systems with the correct volatile compound(s) opens up the opportunity to assist in the battle against many insect-borne diseases. World-wide, and particularly in Africa, many children are dying each day from malaria. The success of the field test with mosquitoes demonstrates that this application against mosquitoes and other insects has the potential of reducing much sickness, suffering and death.

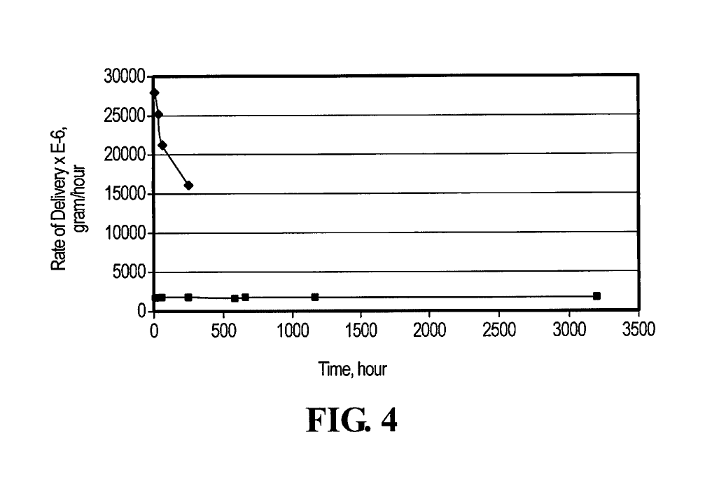

Representative results of laboratory test are reflected in FIG. 4 and confirm that a consistent release rate over a long period of time, displayed by the lower curve, is attained. The upper curve depicts an example of the unsuccessful existing state of the art, which reflects the high rate of release and the short duration of time. Comparison of the two curves demonstrates that the desired requirements can be achieved when using the permeation-based vapor deliver system. Experimental release rates of a volatile compound from a model fixed system were measured as a function of time. Glass screw-thread sample vials with PTFE/silicone septa and open-top polypropylene caps contained the volatile liquid compound. Two different sizes of vials were employed: Small 8 mL capacity (17 mm O.D. and 63 mm Height) and large 40 mL capacity (28 mm O.D. and 98 mm Height). The test volatile compound selected for study was dimethyldisulfide (DMDS). Duplicate samples of 5.5 mL and 30 mL of DMDS were added to the small and large vials, respectively. Then a permeation membrane was placed on the top of each vial, and the open-top cap was tightly screwed to seal the vial contents. The diameters of the membranes for the small and large vials were 13.4 and 22.0 mm, respectively. In addition, the diameters of the openings in the caps for the small and large vials were 8.78 mm and 14.62 mm, respectively. Each vial with membrane, cap and liquid contents was then immediately weighed on an analytical balance. Next, the vials were placed in a dedicated laboratory fume hood. Ambient air with a bulk velocity of about 1.4 m/s (or 3 mph) passed over the vials. The targeted air velocity is representative of the average air velocity at the closest weather station to the planned grove test site over a one-year period. At selected times the vials with membrane, cap and liquid were weighed to obtain the mass loss of the vial contents as the volatile compound permeated through the membrane of the vial over time. From these primary measurements the release rate for each nozzle was determined as a function of time.

Permeation rates were obtained for a variety of membranes including low-density polyethylene (0.004), acrylonitrile butadiene styrene (0.003), polyethylene terephthalate (0.003), polymethylpentene (0.003), polytetrafluoroethylene (0.003), polycarbonate (0.04), acetate (0.010) and acetal copolymer (0.010). The numbers in parentheses represent the membrane thickness stated in inches. In addition, the effect of membrane thickness was examined by using low-density polyethylene (0.004 and 0.010), high-density polyethylene (0.015 and 0.030), and ultrahigh-molecular weight polyethylene (0.005, 0.010 and 0.020).

FIG. 4 shows the results of the release rate performance of a representative membrane (low-density polyethylene, 0.004 inch-thick). The laboratory measurements confirm that a substantially constant release rate (26 mg/day) is attainable over an extremely long period of time. While this test lasted for 133 days in the laboratory, field tests have demonstrated that this rate may continue for over one year.

The upper curve of FIG. 4 depicts an example of the unsuccessful existing state of the art open evaporative process, which reflects the high rate of release and short duration of time Comparison of the two curves demonstrates that dramatically improved results over the known prior art can be achieved when using the permeation-based vapor delivery system of the present invention.

Field tests using the invention were performed starting with the preparation of an abandoned citrus grove that had not been sprayed with pesticides in the previous five years. This citrus grove is located in Orange County, Fla. The grove was prepared by hedging test site trees and fertilizing to promote new growth (flush). When the grove had flushed, four trees in each of three rows were selected and a fixed passive control system was installed for each of three different sized fixed systems. The systems all contained the same selected volatile organosulfur compound. After weekly tap testing and visually observing the leaves with a magnifying glass it was concluded that the volatile sulfur compound that had been in use did not repel the psylllids. However, it was observed that the fixed systems still contained and were dispensing the volatile compound after being in the field for over one year.

Other field tests were performed using a single citrus tree (6 feet high and 4 feet wide) that was heavily infested with psyllids and infected with HLB disease located in a non-pesticide sprayed urban area near Naples, Florida. It was observed that the citrus tree was flushing and then a tap test was performed and significant psyllids were confirmed to be present. Two fixed systems were installed using the same volatile organosulfur compound used in the previously-described field test. Weekly tap tests and visual inspection using a magnifying glass confirmed that the volatile sulfur compound did not repel the psyllids. However, a different volatile organosulfur compound was selected and the test continued. Initial tap test and visual inspection revealed that the tree was heavily infested and weekly evaluations confirmed that this volatile also was not repelling the psyllids. Another, different volatile organosulfur compound was selected and the test continued. Weekly tests and observations showed that while this volatile compound did not completely eliminate the psyllids, it was observed that the test count was reduced by more than an order of magitude. Thereafter, a different volatile organosulfur blend was selected and a single fixed system was installed. Tap tests and observation showed that the tree was still infested, but at a lower level than when the tests initially commenced. However, tap tests reflected the same level of adult psyllid infestation but magnified visual observation showed no eggs or nymphs present. Weekly tests and observations continued with the resulting find of 0 to 3 adult psyllids on all occasions and only one nymph found on two occasions. The average release rate of the selected volatile from the fixed system was 83 mg/day during this test. From these tests it was concluded that using the fixed passive control system this volatile sulfur compounds can be effective in dramatically reducing the adult population of psyllids on a small tree that had been heavily infested initially. This last test continued through two flush periods. Additionally during a five month period only one nymph was found on two occasions, reflecting a very favorable impact on the reproduction of psyllids.

It should be noted that nearby the test tree there was an untreated citrus tree (comparative standard) that was also heavily infested with psyllids and continued to be heavily infested with adults, prolific eggs and nymphs during three flush periods experienced during these tests. The presence of this untreated tree and the observed conditions each week confirm the validity of the results observed on the treated test tree. The fixed passive control system is effective when armed with an appropriate/correct volatile organosulfur compound(s) in a single tree application.

Further field tests were performed to evaluate the fixed passive control system in use to repel mosquitoes at a home in Oakland, Florida. Two fixed passive control systems were hung under the eve of a home over the front door. This home is located in a lovely wooded lot and has hedging along both sides of the front walk extending from the front door out to the drive. The residents have a German Shepard dog that requires walking several times a day and sometimes during the night. Each time they would walk the dog the mosquitoes would attack both human and dog. The results were both resulting bites and bringing mosquitoes in the house on their clothes and on the dog. After hanging the two fixed systems on the eve the mosquitoes decreased to where they were no longer a problem around the front door. To check the presence of mosquitoes a container was allowed to fill with water and was checked for larvae. Mosquitoes were observed in the yard during this time. A third fixed system was added to the two under the eve and the area around the front door continued to not have a mosquito problem. This positive result continued for more than one year.

From these field tests it is concluded that this volatile sulfur compound used in the fixed system is very effective in a residential application to repel mosquitoes. The fixed passive control system is effective in repelling mosquitoes in a residential application with the appropriate volatile organosulfur compound.

Disclosed herein, then, are fixed-supply apparatuses that can function as stand-alone devices and distributed systems that can cover wider areas and more items to be treated/protected/etc. The distributed systems can take the form of pumped systems or gravity-fed systems. Moreover, those skilled in the art will appreciate from this disclosure that the in both the gravity-fed and pumped distributed systems the flow rate is extremely low, such that to the human eye the actual flow might be imperceptible. In this regard, the conduits or pipes delivering the volatile compound to the nozzles can be considered to be containment devices as well, given the extremely low flow rates.

While the invention has been described with reference to preferred and example embodiments, it will be understood by those skilled in the art that a variety of modifications, additions and deletions are within the scope of the invention, as defined by the following claims.

What is claimed is:

1. An apparatus for a long-term sustained application of a controlled amount of a volatile compound to an open outdoor area or to a similarly open indoor area, the apparatus comprising:
a compound-dispensing structure for containing a volume of the volatile compound, a permeable membrane at least partially capping the volume of volatile compound and permitting the volatile compound to pass therethrough by permeation at a substantially constant rate, and at least partially open cover having openings formed therein and being positioned over the permeable membrane.

2. An apparatus as claimed in claim 1 wherein the membrane is a polymeric membrane.

3. An apparatus as claimed in claim 1 wherein the volatile compound comprises a pesticide.

4. An apparatus as claimed in claim 3 wherein the volatile compound comprises a naturally-occurring pesticide.

5. An apparatus as claimed in claim 1 wherein the volatile compound comprises a pest-repellant.

6. An apparatus as claimed in claim 5 wherein the volatile compound comprises a naturally-occurring pest-repellant.

7. An apparatus as claimed in claim 1 wherein the volatile compound comprises a naturally-occurring attractant for beneficially attracting insects.

8. An apparatus as claimed in claim 1, wherein the compound-dispensing structure comprises a non-permeable reservoir.

9. The apparatus of claim 1, wherein the permeable membrane is flat.

10. The apparatus of claim 1, wherein the permeable membrane is cylindrical.

11. The apparatus of claim 1, comprising a plurality of the structures adapted and configured as fixed-supply reservoirs.

12. The apparatus of claim 1, comprising a plurality of the structures adapted and configured as pumped-supply reservoirs and wherein the permeable membranes function as passive flow control nozzles.

13. The apparatus of claim 12, further comprising a main supply reservoir for containing a supply of the volatile compound and a plurality of conduits extending to the nozzles for delivering the volatile compound from the main supply to the passive flow control nozzles.

14. The apparatus of claim 13, further comprising one or more pumps for pumping the volatile compound through the conduits to the passive flow control nozzles.

15. The apparatus of claim 13, wherein the passive flow control nozzles for dispensing the volatile compound dispense the volatile compound at a substantially constant low flow rate.

16. The apparatus of claim 14, wherein the apparatus is adapted to provide pesticide to fields of plants and/or trees.

17. The apparatus of claim 14, wherein the apparatus is adapted to provide naturally-occurring pest-repellant to groves of plants and/or trees.

18. The apparatus of claim 14, wherein the apparatus is adapted to provide naturally-occurring attractant for beneficially attracting insects to groves of plants and/or trees.

19. A system for a long-term sustained application of a controlled amount of a volatile compound to an open outdoor area or to a similarly open indoor area, the system comprising:
a plurality of spaced-apart compound-dispensing nodes, each compound-dispensing node comprising a permeable membrane permitting the volatile compound to pass therethrough by permeation at a substantially continuous rate;
a main supply reservoir for containing a supply of the volatile compound; and
a plurality of conduits extending to the membranes for delivering the volatile compound from the main supply to the membranes.

20. The system of claim 19, further comprising one or more pumps for pumping the volatile compound through the conduits to the membranes.

21. The system of claim 19, wherein the permeable membranes function as passive flow control nozzles for dispensing the volatile compound at a substantially constant low flow rate over an extended period of time.

22. The system as claimed in claim 21 wherein the membranes are permeable polymeric membranes.

23. The system of claim 19 wherein the volatile compound comprises a pesticide.

24. The system of claim 19 wherein the volatile compound comprises a naturally-occurring pesticide.

25. The system of claim 19 wherein the volatile compound comprises a pest-repellant.

26. The system of claim 19 wherein the volatile compound comprises a naturally-occurring pest-repellant.

27. The system of claim 19 wherein the volatile compound comprises a naturally-occurring attractant for beneficially attracting insects.

28. The system of claim 19 wherein the main supply of volatile compound is positioned at an elevation higher than the permeable membranes such that the volatile compound can flow from the main supply to the permeable membranes by gravity.

29. The system of claim 19 wherein the plurality of conduits extend in a grid-like pattern within a field of trees or plants.

30. The system of claim 19 wherein the plurality of conduits extend around the perimeter of a grove of trees or plants.

31. A method for effecting a controlled release of a volatile compound over time at or adjacent a desired location, comprising the steps of:
providing a reservoir for containment of a volatile compound to be released, the reservoir including a permeation membrane;
placing the volatile compound within the reservoir; and
positioning the reservoir at or adjacent the desired location.

32. The method of claim 31, wherein the release of a volatile compound is into an open local environment.

33. The method of claim 31, wherein the release of a volatile compound is into an open field environment.

34. The method of claim 31, wherein the volatile compound comprises a pesticide.

35. The method of claim 31, wherein the volatile compound comprises a pest repellent.

36. The method of claim 31, wherein the volatile compound comprises a beneficial insect attractant for attracting insects.

37. The method of claim 31, wherein the volatile compound provides enhanced plant immune responses.

38. The method of claim 31, wherein the volatile compound comprises a fumigant.

39. The method of claim 31, wherein a plurality of containers containing the volatile compound are placed in a plurality of trees forming a peripheral border.

40. The method of claim 31 wherein the volatile compound is effective for repelling or killing mosquitoes and other insects and wherein the reservoir is placed near structures or places frequented by humans.

41. A method of treating a citrus tree at risk of disease, infection, or infestation, etc., the method comprising the steps of:
placing a quantity of a selected volatile compound in a container, the container having a permeable membrane adapted and configured to allow the volatile compound to permeate therethrough at a low, substantially constant rate over a long period of time; and
attaching the container to the citrus tree to apply the volatile compound to the citrus tree and to its immediate surroundings and leaving the container on the citrus tree for two months or more to deliver the volatile compound to the citrus tree for two months or longer.

42. The method of claim 41, wherein the volatile compound comprises a pesticide.

43. The method of claim 41, wherein the volatile compound comprises a pest repellent.

44. The method of claim 41, wherein the volatile compound comprises a beneficial insect attractant for beneficially attracting insects.

45. The method of claim 41, wherein the volatile compound provides enhanced plant immune responses.

46. The method of claim 41, wherein the volatile compound comprises a fumigant.

47. The method of claim 41, wherein after a time a second container containing a volatile compound is placed in the tree.

48. The method of claim 41, wherein the container is left on the citrus tree a year or more to deliver the volatile compound to the citrus tree for over a year.

49. The method of claim 41, wherein psyllid control is accomplished using a volatile compound that releases a volatile repellent which is behaviorally efficacious over a growing season to repel psyllid.
